SEQUENCE LISTING (23 sequences)

SEQ ID NO: 1 - protein, 572 aa, C. jejuni
MGFRINTNVAALNAKANADLNSKSLDASLSRLSSGLRINSAADDASGMAIADTLRSQANTLGQAISNGNDAIGILQTADKAMDEQLKILDTIKTKATQAAQDGQSLKTRTMLQADINRLMEELDNIANTTSFNGKQLLSGNFINQEFQIGASSNQTVKATIGATQSSKIGLTRFETGGRISTSGEVQFTLKNYNGIDDFQFQKVVISTSVGTGLGALADEINKNADKTGVRATFTVETRGIAAVRAGATSDTFAINGVKIGKVDYKDGDANGALVAAINSVKDTTGVEASIDANGQLLLTSREGRGIKIDGNIGGGAFINADMKENYGRLSLVKNDGKDILISGSNLSSAGFGATQFISQASVSLRESKGQIDANIADAMGFGSANKGVVLGGYSSVSAYMSSAGSGFSSGSGYSVGSGKNYSTGFANAIAISAASQLSTVYNVSAGSGFSSGSTLSQFATMKTTAFGVKDETAGVTTLKGAMAVMDIAETATTNLDQIRADIGSVQNQVTSTINNITVTQVNVKAAESQIRDVDFAAESANYSKANILAQSGSYAMAQANSVHQNVLRLLQ

SEQ ID NO: 2 - protein, 510 aa, H. pylori
MAFQVNTNINAMNAHVQSALTQNALKTSLERLSSGLRINKAADDASGMTVADSLRSQASSLGQAIANTNDGMGIIQVADKAMDEQLKILDTVKVKATQAAQDGQTTESRKAIQSDIVRLIQGLDNIGNTTTYNGQALLSGQFTNKEFQVGAYSNQSIKASIGSTTSDKIGQVRIATGALITASGDISLTFKQVDGVNDVTLESVKVSSSAGTGIGVLAEVINKNSNRTGVKAYASVITTSDVAVQSGSLSNLTLNGIHLGNIADIKKNDSDGRLVAAINAVTSETGVEAYTDQKGRLNLRSIDGRGIEIKTDSVSNGPSALTMVNGGQDLTKGSTNYGRLSLTRLDAKSINVVSASDSQHLGFTAIGFGESQVAETTVNLRDVTGNFNANVKSASGANYNAVIASGNQSLGSGVTTLRGAMVVIDIAESAMKMLDKVRSDLGSVQNQMISTVNNISITQVNVKAAESQIRDVDFAEESANFNKNNILAQSGSYAMSQANTVQQNILRLLT

SEQ ID NO: 3 - protein, 379 aa, V. cholerae
MTINVNTNVSAMTAQRYLTKATGELNTSMERLSSGNRINSAKDDAAGLQISNRLTAQSRGLDVAMRNANDGISIAQTAEGAMNESTSILQRMRDLALQSANGTNSASERQALNEESVALQDELNRIAETTSFGGRKLLNGSFGEASFQIGSSSGEAIIMGLTSVRADDFRMGGQSFIAEQPKTKEWGVPPTARDLKFEFTKKDGEAVVLDIIAKDGDDIEELATYINGQTDLFKASVDQEGKLQIFVAEPNIEGNFNISGGLATELGLNGGPGVKTTVQDIDITSVGGSQNAVGIIDAALKYVDSQRADLGAKQNRLSHSISNLSNIQENVEASKSRIKDTDFAKETTQLTKSQILQQAGTSILAQAKQLPNSAISLLQ

SEQ ID NO: 4 - protein, 394 aa, P. aeruginosa
MALTVNTNIASLNTQRNLNNSSASLNTSLQRLSTGSRINSAKDDAAGLQIANRLTSQVNGLNVATKNANDGISLAQTAEGALQQSTNILQRMRDLSLQSANGSNSDSERTALNGEAKQLQKELDRISNTTTFGGRKLLDGSFGVASFQVGSAANEIISVGIDEMSAESLNGTYFKADGGGAVTAATASGTVDIAIGITGGSAVNVKVDMKGNETAEQAAAKIAAAVNDANVGIGAFSDGDTISYVSKAGKDGSGAITSAVSGVVIADTGSTGVGTAAGVAPSATAFAKTNDTVAKIDISTAKALSRRAGDRTTAIKQIDASVPTSVAVQNRFDNTINNLKNIGENVSAARGRIEDTDFAAETANLTKNQVLQQAGTAILAQANQLPQSVLSLLR

SEQ ID NO: 5 - protein, 170 aa, R. sphaeroides
MTTINTNIGAIAAQANMTKVNDQFNTAMTRLSTGLRINAAKDDAAGMAIGEKMTAQVMGLNQAIRNAQDGKNLVDTTEGAHVEVSSMLQRLRELAVQSSNDTNTAADRGSLAAEGKQLIAEINRVAESTTFNGMKVLDGSFTGKQLQIGADSGQTMAINVDSAAATDIGA

SEQ ID NO: 6 - protein, 365 aa, P. mirabilis1
MAQVINTNYLSLVTQNNLNKSQGTLGSAIERLSSGLRINSAKDDAAGQAIANRFTSNVNGLTQASRNANDGISIAQTTEGALNEINNNLQRIRELTVQAKNGTNSNSDITSIQNEVKNVLDEINRISEQTQFNGVKVLSGEKSEMVIQVGTNDNETIKFNLDKVDNDTLGVASDKLFDTKTEKKGVTAAGAGVTDAKKINAAATLDMMVSLVKEFNLDGKPVTDKFIVTKGGKDYVATKSDFELDATGTKLGLKASATTEFKVDAGKDVKTLNVKDDALATLDKAINTIDESRSKLGAIQNRFESTINNLNNTVNNLSASRSRILDADYATEVSNMSRGQILQQAGTSVLAQANQVPQTVLSLLR

SEQ ID NO: 7 - protein, 367 aa, P. mirabilis2
MAQVINTNYLSLVTQNNLNRSQSALGNAIERLSSGMRINSAKDDAAGQAIANRFTSNINGLTQASRNANDGISVSQTTEGALNEINNNLQRIRELTVQAKNGTNSNSDINSIQNEVNQRLDEINRVSEQTQFNGVKVLSGEKSKMTIQVGTNDNEVIEFNLDKIDNDTLGVASDKLFDAKTEKKGVTAAGDAIDANALGISGSKKYVTGISVKEYKVDGKVSSDKVVLNDGSDDYIVSKSDFTLKSGTTTGEVEFTGSKTTKFTADAGKDVKVLNVKDDALATLDNAISKVDESRSKLGAIQNRFQSTINNLNNTVNNLSASRSRILDADYATEVSNMSKNQILQQAGTAVLAQANQVPQTVLSLLR

SEQ ID NO: 8 - protein, 506 aa, S. typhimurium2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 9 - protein, 490 aa, S. typhimurium1
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVNGQTQFSGVKVLAQDNTLTIQVGANDGETIDIDLKQINSQTLGLDTLNVQQKYKVSDTAATVTGYADTTIALDNSTFKASATGLGGTDEKIDGDLKFDDTTGKYYAKVTVTGGTGKDGYYEVSVDKTNGEVTLAAVTPATVTTATALSGKMYSANPDSDIAKAALTAAGVTGTASVVKMSYTDNNGKTIDGGLAVKVGDDYYSATQDKDGSISIDTTKYTADNGTSKTALNKLGGADGKTEVVTIDGKTYNASKAAGHDFKAEPELAEQAAKTTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 10 - protein, 351 aa, S. marcesens
MAQVINTNSLSLMAQNNLNKSQSSLGTAIERLSSGLRINSAKDDAAGQAISNRFTANIKGLTQASRNANDGISLAQTTEGALNEVNDNLQNIRRLTVQAQNGSNSTSDLKSIQDEITQRLSEINRISEQTDFNGVKVLSSDQKLTIQVGANDGETTDIDLKKIDAKQLGMDTFDVTTKSAKAGAEIATGTKITVDSDATKQADADVTGLAKGQTLVSGTDADGKSAYFIATKDDATGDVAYTKAKVADDGKVTDSGTDAGVKNPLATLDKALAQVDGLRSSLGAVQNRFDSVINNLNSTVNNLSASQSRIQDADYATEVSNMSRANILQQAGTSVLAQANQSTQNVLSLLR

SEQ ID NO: 11 - protein, 554 aa, E. coli
MAQVINTNSLSLITQNNINKNQSALSSSIERLSSGLRINSAKDDAAGQAIANRFTSNIKGLTQAARNANDGISVAQTTEGALSEINNNLQRIRELTVQATTGTNSDSDLDSIQDEIKSRLDEIDRVSGQTQFNGVNVLSKDGSMKIQVGANDGETITIDLKKIDSDTLNLAGFNVNGEGETANTAATLKDMVGLKLDNTGVTTAGVNRYIADKAVASSTDILNAVAGVDGSKVSTEADVGFGAAAPGTPVEYTYHKDTNTYTASASVDATQLAAFLNPEAGGTTAATVSIGNGTTAQEQKVIIAKDGSLTAADDGAALYLDDTGNLSKTNAGTDTQAKLSDLMANNANAKTVITTDKGTFTANTTKFDGVDISVDASTFANAVKNETYTATVGVTLPATYTVNNGTAASAYLVDGKVSKTPAEYFAQADGTITSGENAATSKAIYVSANGNLTTNTTSESEATTNPLAALDDAIASIDKFRSSLGAIQNRLDSAVTNLNNTTTNLSEAQSRIQDADYATEVSNMSKAQIIQQAGNSVLAKANQVPQQVLSLQQG

SEQ ID NO: 12 - protein, 550 aa, S. flexneri
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGGGAVANTAASKADLVAANATVVGNKYTVSAGYDAAKASDLLAGVSDGDTVQATINNGFGTAASATNYKYDSASKSYSFDTTTASAADVQKYLTPGVGDTAKGTITIDGSAQDVQISSDGKITASNGDKLYIDTTGRLTKNGSGASLTEASLSTLAANNTKATTIDIGGTSISFTGNSTTPDTITYSVTGAKVDQAAFDKAVSTSGNNVDFTTAGYSVNGTTGAVTKGVDSVYVDNNEALTTSDTVDFYLQDDGSVTNGSGKAVYKDADGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQG

SEQ ID NO: 13 - protein, 286 aa, T. pallidumA
MIINHNMSAMFAQRTLGHTNVQVGKGIEKLSSGYRINRAGDDASGLAVSEKMRSQIRGLNQASTNASNGVNFIQVTEAYLQETTDIMQRIRELAIQAANGIYSAEDRMQIQVEVSQLVAEVDRIASSAQFNGMNLLTGRFSRTEGENVIGGSMWFHIGANMDQRMRVYIGTMTAVALGVRNGVDESIMSIETADSANKSIGTIDAALKRINKQRADLGGYQNRMEYTVVGLDIAAENLQAAESRIRDANIAKQMVEYTKNQVLTQSGTAMLAQANTSAQSILSILR

SEQ ID NO: 14 - protein, 286 aa, T. pallidumB
MIINHNMSAMFAQRTLGNTNLSVQKNMEKLSSGLRINRAGDDASGLAVSEKMRSQIRGLNQASTNAQNGISFIQVAESYLQETTDVIQRIRELSVQSANGIYSAEDRMYIQVEVSQLVAEIDRIASHAQFNGMNMLTGRFARETGENTVTASMWFHIGANMDQRTRAYIGTMTAAALGVRDVGDESILNIDDPEKANRAIGTLDEAIKKINKQRADLGAYQNRLEYTVIGVNVAAENLQAAESRIRDVDMAKEMVDYTKNQILVQSGTAMLAQANQATQSVLSLLR

SEQ ID NO: 15 - protein, 283 aa, L. pneumophila
MIINHNLSAVNAHRSLKFNELAVDKTMKALSSGMRINSAADDASGLAVSEKLRTQVNGLRQAERNTEDGMSFIQTAEGFLEQTSNIIQRIRVLAIQTSNGIYSNEDRQLVQVEVSALVDEVDRIASQAEFNKFKLFEGQFARGSRVASMWFHMGPNQNQRERFYIGTMTSKALKLVKADGRPIAISSPGEANDVIGLADAALTKIMKQRADMGAYYNRLEYTAKGLMGAYENMQASESRIRDADMAEEVVSLTTKQILVQSGTAMLARANMKPNSVLKLLQHI

SEQ ID NO: 16 - protein, 336 aa, B. burgdorferei
MIINHNTSAINASRNNGINAANLSKTQEKLSSGYRINRASDDAAGMGVSGKINAQIRGLSQASRNTSKAINFIQTTEGNLNEVEKVLVRMKELAVQSGNGTYSDADRGSIQIEIEQLTDEINRIADQAQYNQMHMLSNKSASQNVRTAEELGMQPAKINTPASLSGSQASWTLRVHVGANQDEAIAVNIYAANVANLFSGEGAQAAQTAPVQEGAQQEGAQQPAPVTAPSQGGVNSPVNVTTTVDANTSLAKIENAIRMISDQRANLGAFQNRLESIKDSTEYAIENLKASYAQIKDATMTDEVVAATTNSILTQSAMAMIAQANQVPQYVLSLLR

SEQ ID NO: 17 - protein, 304 aa, B. subtilus
MRINHNIAALNTLNRLSSNNSASQKNMEKLSSGLRINRAGDDAAGLAISEKMRGQIRGLEMASKNSQDGISLIQTAEGALTETHAILQRVRELVVQAGNTGTQDKATDLQSIQDEISALTDEIDGISNRTEFNGKKLLDGTYKVDTATPANQKNLVFQIGANATQQISVNIEDMGADALGIKEADGSIAALHSVNDLDVTKFADNAADTADIGFDAQLKVVDEAINQVSSQRAKLGAVQNRLEHTINNLSASGENLTAAESRIRDVDMAKEMSEFTKNNILSQASQAMLAQANQQPQNVLQLLR

SEQ ID NO: 18 - protein, 281 aa, C. difficile
MRVNTNVSALIANNQMGRNVSGQSKSMEKLSSGLRIKRAADDAAGLAISEKMRAQLKGLDQAGRNVQDGISVVQTAEGALEETGNILTRMRTLAVQASNETNSKDERAKIAGEMEQLRSEVDRIADSTKFNGENLLSSDKKIALQVGAEAVSNNVIEVSLINTKGVLTTRNVNSANIDAMSVSGSIGTEAASKMIVNLDSSLADINSARALLGAQQNRLESTQNNLNNTVENVTAAESRIRDTDVASEMVNLSKMNILVQASQSMLSQANQQPQGVLQLLG

SEQ ID NO: 19 - protein, 394 aa, R. meliloti
MTSILTNNSAMAALSTLRSISSSMEDTQSRISSGLRVGSASDNAAYWSIATTMRSDNQALSAVQDALGLGAAKVDTAYSGMESAIEVVKEIKAKLVAATEDGVDKAKIQEEITQLKDQLTSIAEAASFSGENWLQADLSGGPVTKSVVGGFVRDSSGAVSVKKVDYSLNTDTVLFDTTGNTGILDKVYNVSQASVTLPVNVNGTTSEYTVGAYNVDDLIDASATFDGDYANVGAGALAGDYVKVQGSWVKAVDVAATGQEVVYDDGTTKWGVDTTVTGAPATNVAAPASIATIDITIAAQAGNLDALIAGVDEALTDMTSAAASLGSISSRIDLQSDFVNKLSDSIDSGVGRLVDADMNEESTRLKALQTQQQLAIQALSIANSDSQNVLSLFR

SEQ ID NO: 20 - protein, 306 aa, A. tumefaciens
MASILTNNNAMAALSTLRSIASDLSTTQDRISSGLKVGSASDNAAYWSIATTMRSDNKALGAVSDALGMGAAKVDTASAGMDAAIKVVTDIKAKVVAAKEQGVDKTKVQEEVSQLLDQLKSIGTSASFNGENWLVSSANATKTVVSGFVRDAGGTVSVKTTDYALDANSMLYTEGTPGTIDANSGILNATGATTTVGAKTYTQISVLDMNVGTDDLDNALYSVETALTKMTSAGAKLGSLSARIDLQSGFADKLSDTIEKGVGRLVDADMNEESTKLKALQTQQQLAIQALSIANSDSQNILSLFR

SEQ ID NO: 21 - protein, 410 aa, R. lupini
MASVLTNINAMSALQTLRSISSNMEDTQSRISSGMRVGSASDNAAYWSIATTMRSDNASLSAVQDAIGLGAAKVDTASAGMDAVIDVVKQIKNKLVTAQESSADKTKIQGEVKQLQEQLKGIVDSASFSGENWLKGDLSTTTTKSVVGSFVREGGTVSVKTIDYALNASKVLVDTRATGTKTGILDTAYTGLNANTVTVDINKGGVITQASVRAYSTDEMLSLGAKVDGANSNVAVGGGSAFVKVDGSWVKGSVDAAASITASTPVAGKFAAAYTAAEAGTAAAAGDAIIVDETNSGAGAVNLTQSVLTMDVSSMSSTDVGSYLTGVEKALTSLTSAGAELGSIKQRIDLQVDFASKLGDALAKGIGRLVDADMNEESTKLKALQTQQQLAIQSLSIANSDSQNILSLFR

SEQ ID NO: 22 - protein, 287 aa, L. monocytogenes
MKVNTNIISLKTQEYLRKNNEGMTQAQERLASGKRINSSLDDAAGLAVVTRMNVKSTGLDAASKNSSMGIDLLQTADSALSSMSSILQRMRQLAVQSSNGSFSDEDRKQYTAEFGSLIKELDHVADTTNYNNIKLLDQTATGAATQVSIQASDKANDLINIDLFNAKGLSAGTITLGSGSTVAGYSALSVADADSSQEATEAIDELINNISNGRALLGAGMSRLSYNVSNVNNQSIATKASASSIEDADMAAEMSEMTKYKILTQTSISMLSQANQTPQMLTQLINS

SEQ ID NO: 23 - protein, 399 aa, B. clarridgeiae
MGTSLLTNKSAMTALQTLRSIDANLDRSKDRVSTGLRISNASENTAYWSISSMMRHDSNTMSAIVDAINLGKEQVGIADTAIGLTKEALDDIQKSMVSAREKGSDDIAKIQDSIIGNMKNISNAVQSASFGGKNILSNGGQTVGMAAGYRREGTAVYVDMIDVGGSELNFGTIGSDGTIDMSQGVLGGIFGTSKGDEGEDVVGKGIGAFSAAHATYKGLEDTLRNAEADLAKAIAKYGESPEDEPGKAIIEKAKQAVETAKTGLKDGQEAYNKAKGEFQTVLDGMTLADFTELKGLGELHSDIQRMIMTSVQNTVRDAVNVTLTAGSKIGAAVNLVNIQLNFVKKLLDNVEVGIGALVDADMNAESAKLAALQVQQQLGIQALSIANQGSQNILALFRN

(19) United States
(12) Patent Application Publication
(10) Pub. No.: US 2009/0297552 A1
(43) Pub. Date: Dec. 3, 2009

(54) FLAGELLIN POLYPEPTIDE VACCINES

(76) Inventors: Alan A. Aderem, Seattle, WA (US); Edward A. Miao, Seattle, WA (US); Carrie M. Rosenberger, Seattle, WA (US)

Correspondence Address: MORRISON & FOERSTER LLP, 12531 HIGH BLUFF DRIVE, SUITE 100, SAN DIEGO, CA, 92130-2040, US

(21) Appl. No.: 12/429,971

(22) Filed: Apr. 24, 2009

Related U.S. Application Data
(60) Provisional application No. 61/048,100, filed on Apr. 25, 2008.

(51) Int. Cl. A61K 39/00; A61K 39/12; A61K 39/02

(52) U.S. Cl. 424/192.1; 424/205.1; 424/235.1

(57) ABSTRACT

Vaccines that comprise or generate immunomodulatory flagellin polypeptides able to stimulate an innate immune response intracellularly and extracellularly employ viruses, bacteria or parasitic cells that contain expression systems for such polypeptides, as well as fusion proteins that contain antigens and/or cell penetrating peptides along with the immunomodulatory peptide.

### CROSS-REFERENCE TO RELATED APPLICATIONS

This application claims benefit under 35 U.S.C. § 119(e) of U.S. Ser. No. 61/048,100 filed 25 Apr. 2008. The contents of this application are incorporated herein by reference.

### ACKNOWLEDGEMENT OF GOVERNMENT SUPPORT/STATEMENT OF RIGHTS TO INVENTIONS MADE UNDER FEDERALLY SPONSORED RESEARCH

This invention was made in part with support from the U.S. government under National Institutes of Health Contracts R01 A1052286 and K08 AI065878. The U.S. government has certain rights in this invention.

### REFERENCE TO SEQUENCE LISTING SUBMITTED VIA EFS-WEB

The entire content of the following electronic submission of the sequence listing via the USPTO EFS-WEB server, as authorized and set forth in MPEP §1730 II.B.2(a)(C), is incorporated herein by reference in its entirety for all purposes. The sequence listing is identified on the electronically filed text file as follows:

| File Name | Date of Creation | Size (bytes) |
|---|---|---|
| 655652000300Seqlist.txt | Apr. 23, 2009 | 68,605 bytes |

### TECHNICAL FIELD

The invention relates to vaccines that provide flagellin polypeptides for stimulation of an innate immune response. The flagellin polypeptides may be used alone or in conjunction with antigens for eliciting adaptive immune responses.

### BACKGROUND ART

Flagellin is an approximately 500 amino acid monomeric protein that polymerizes to form the flagella associated with bacterial motion. Flagella are whip-like structures that enable bacterial motility by propeller-like rotation. These structures are polymers consisting of flagellin and are anchored to the bacterial cell wall by a basal body and hook structure. The term “flagellin” refers to the monomer subunit that polymerizes to form the filament, while the term “flagellar” refers more generally to any component of the filament, basal body or hook.

Flagellar gene expression is tightly regulated; the hook and basal body (HBB) genes are expressed first and the final flagellar components are expressed only when the HBB is completely assembled. Flagellar genes are divided into three transcriptional classes. Class I genes include the master transcriptional regulatory proteins FlhC/FlhD. Class II genes encode the basal body and hook, and also include the transcriptional activator FliA, and the FliA repressor FlgM. Upon completion of the HBB, the repressor FlgM is exported through the HBB. This depletes the bacterial cytosol of FlgM protein, thus releasing FliA, which binds to class III gene promoters and activates their transcription. Class III genes encode the hook-filament adaptors, cap, motor, chemosensory system, and the flagellin protein that polymerizes to form the flagellar filament.

Flagellin is exported by a type III secretion system (T3SS) located in the HBB that is evolutionarily related to other T3SS that transport virulence factors. This secretory apparatus forms a single structure that spans the inner membrane, periplasmic space and outer membrane, terminating in a hook structure on the exterior of the bacterial cell wall. Flagellin is exported through the hollow core of the HBB, where up to 30,000 flagellin subunits assemble at the end of the hook.

The amino acid sequences of flagellin from various bacterial species are set forth in SEQ ID NO:1-SEQ ID NO:23. The nucleotide sequences encoding the listed flagellin polypeptides are also publicly available in the NCBI GenBank database. The flagellin sequences from S. Typhimurium, H. Pylori, V. Cholera, S. marcesens, S. flexneri, T. Pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli, among others are known.

The flagellin monomer is shaped like the capital Greek letter gamma (Γ) and is formed by domains D0 through D3. D0 and D1, which form the stem, are composed of tandem long alpha helices and are highly conserved among different bacteria. When the monomer is stacked, D0 and D1 are buried in the center of the filament. The top of the Γ is composed of D2 and D3, two highly variable globular domains that are exposed on the surface of the flagellar filament and to which antibody responses are directed. The D2 and D3 domains, however, are not involved in eliciting an innate immune response.

Innate immune responses are mediated by toll-like receptors (TLR's) at cell surfaces and by Nod-LRR proteins (NLR) intracellularly and are mediated by D1 and D0 regions respectively. The innate immune response includes cytokine production in response to TLR (including TLR5) activation and activation of Caspase-1 and IL-1β secretion in response to certain NLRs (including Ipaf). This response is independent of specific antigens, but can act as an adjuvant to an adaptive immune response that is antigen specific. The antigen may be supplied externally in the form of a vaccine or infection, or may be indigenous, for example, as is the case for tumor-associated antigens.

PCT publication WO02/085933 published 31 Oct. 2002 demonstrates that flagellar polypeptides are able to stimulate an innate immune response through interaction with the toll-like receptor 5 (TLR5). This receptor, which is displayed on cell surfaces, interacts with the flagellin polypeptide extracellularly. U.S. patent publication 2005/0147627 published 7 Jul. 2005 notes that the region of flagellin responsible for interaction with TLR5 is found only in the D1 domain. Smith, K. D., et al., Nature Immunol. (2003) 4:1247-1253 disclose that TLR5 recognizes a site on the flagellin of Salmonella typhimurium composed of N-terminal residues 78-129 and 135-173 and C-terminal residues 395-444.

It was subsequently found that cytoplasmic flagellin activates Caspase 1 and effects secretion of interleukin 1β via Ipaf, which is also designated NLRC4. Miao, E. A., et al., Nat. Immunol. (2006) 7:569-575; Miao, E. A., et al., Semin. Immunopathol. (2007) 29:275-288. The region of flagellin responsible for this activation appears to be the C-terminal 35 amino acids in the D0 region of the flagellin of Legionella pneumophila at positions 441-475 which are sufficient for activation of NLRC4 which is enhanced by a functional NLR-apoptosis inhibitory protein 5 (Naip5). Lightfield, K. L., et al., Nature Immunol. (2008) 9:1171-1178.

Although use of these polypeptides in vaccines has been described generally, there is no suggestion of vaccines that will provide both intracellular and extracellular responses to the flagellin polypeptides; nor are fusion peptides comprising an immunomodulatory flagellin polypeptide fused to a desired antigen described. Thus, the present invention is directed to improvements in vaccines that employ flagellin polypeptides to elicit an innate immune response.

Any documents cited in this Background section and throughout the specification are hereby incorporated herein by reference in their entirety, as are amino acid and nucleotide sequences of peptides/proteins referred to herein and their corresponding ORFs available in publications and public databases.

### DISCLOSURE OF THE INVENTION

The invention is directed to improved vaccines that employ flagellin polypeptides able to elicit both extracellular and intracellular based innate immune responses and to vaccines that comprise fusion proteins that are composed of the immunomodulatory flagellin polypeptide coupled to a desired antigen and/or to a sequence that facilitates cellular uptake.

Because both bacteria and viruses are able to invade cells, and because bacteria can effectively secrete flagellin proteins when provided signal sequences and through endogenous secretion mechanisms into cells that are destined for infection, vaccines based on modified viruses and bacteria that contain genetic constructs for the production of the flagellin polypeptides are included in the invention. In addition, mediators of protein transfection of cells, such as listeriolysin O and other transfection agents such as tat proteins and melittin provide a means for delivery of flagellin into the cytoplasm. This last approach has been described in in vitro studies by Amer, A., et al., J. Biol. Chem. (2006) 281:35217-35223; Franchi, L., et al., Nat. Immunol. (2006) 7:576-582; Miao, E. A., et al., Nat. Immunol. (2006) 7:569-575; Molofsky, A. B., et al., J. Exp. Med. (2006) 203:1093-1104; and Wren, T., et al., PLoS Pathog. (2006) 2:e18. The present invention also includes protein-based vaccines that provide transfection reagents that are pharmacologically acceptable.

Thus, in one aspect, the invention is directed to compositions containing recombinant constructs for the production of flagellin polypeptides which are able to generate either an extracellular-based response to the flagellin polypeptide or an intracellular response to a flagellin polypeptide or both. Various alternative embodiments within this general concept are envisioned.

In one embodiment, a nucleotide sequence encoding the D0 or D1 region of a flagellin polypeptide or both is inserted into the genome of an attenuated virus, such as an influenza virus. Included within this embodiment are vaccines intended to target only the intracellular receptor and wherein the nucleotide sequence may encode only the D0 region of a flagellin monomer, embodiments wherein only the D1 region is encoded which activate the external receptor, or wherein both the D0 and D1 regions are encoded thus activating both.

In another embodiment, an attenuated bacterial strain is employed having nucleotide sequences encoding D1 and/or D0 regions of the flagellin monomer or both contained in an expression system that operates extrachromosomally or from within the genome. Cells of eukaryotic parasites may also be used.

In a third embodiment, the relevant flagellin monomer is administered along with a non-toxic transfection reagent, either as separate moieties or as a fusion protein.

In still another embodiment, the relevant portions of the flagellin polypeptide are coupled to a desired antigen. In this embodiment, as well, the D0 and/or D1 regions are included.

Other aspects are a method to treat or reduce the risk of a pathogenic infection or disease in a mammal, comprising administering to the mammal a composition of the invention.

### BRIEF DESCRIPTION OF THE DRAWINGS

FIGS. 1A and 1B show the results of experiments conducted with a modified S. typhimurium containing the FliC gene from an SPI2 regulated promoter in pWSK29. FIG. 1A shows the ability of these modified cells to produce flagellin as determined by IL-1b secretion in macrophage that are wildtype or that lack Ipaf. FIG. 1B shows the bacterial counts of spleen and liver in wildtype and Ipaf null mice infected with these bacteria.

### MODES OF CARRYING OUT THE INVENTION

The present invention relates to live or replication-competent vaccine compositions, and methods of using the same, comprising, for example, a virus, a bacteria, or a eukaryotic parasitic organism, wherein the vaccine composition comprises a nucleotide sequence that encodes, and endogenously expresses, an immunomodulatory flagellin polypeptide. In certain embodiments, the live vaccine composition is attenuated, such that it replicates in the mammal, and thereby generates a broad and effective immune response, but typically does not cause a pathological infection.

The immunomodulatory flagellin polypeptides provided herein function generally to stimulate and/or enhance an innate immune response, which thereby stimulates and/or enhances an adaptive immune response (i.e., the humoral and cell-mediated immune responses). An enhanced immune response causes a general increase in immune system activity that can result in the destruction of foreign or pathologically aberrant cells that otherwise would have escaped the immune response. In certain embodiments, the endogenously expressed flagellin polypeptides of the present invention stimulate toll-like receptor 5 (TLR5) and Ipaf, both of which mediate certain aspects of the innate immune response, such as by regulating the expression and secretion of various immune regulatory cytokines.

### DEFINITIONS

As used in this application, the singular forms “a,” “an” and “the” include plural references unless the content clearly dictates otherwise.

By “isolated” is meant material that is substantially or essentially free from components that normally accompany it in its native state.

The terms “replication-competent” or “live” refers generally to a virus, bacteria, or parasite that is capable of more than one round of division or expansion a host or a host cell. For example, a replication-competent virus is generally capable of replicating or expanding beyond a single round of infection in a population of cells (e.g., in a cell culture or in an organism), such as by infecting a first cell and producing one or more virus particles within that first cell that are capable of infecting additional cells, and so on. A live bacteria is generally be able to undergo multiple cell divisions, thereby producing daughter cells from a parent cell, which typically undergo further cell division.

The term “attenuated” refers generally to a virus, bacteria, or parasite that is capable of replicating or undergoing cell division within a host, but is not significantly pathogenic to the host (i.e., does not cause a significant “pathological condition”). An attenuated vaccine may be prepared from live microorganisms or viruses cultured under adverse conditions leading to loss of their virulence or pathogenicity but retention of their ability to induce protective immunity, or by removing certain non-essential genes (e.g., genes that are non-essential for replication or cell division) that would otherwise contribute to the pathogenesis or virulence of the microorganism or virus.

The term “prevent” or “preventive” or “prophylactic” relates to “reducing the risk” in acquiring a disease or pathological condition, such as a microbial or viral infection or cancerous condition. The term “prevent” does not necessarily remove all risk of acquiring a give disease or condition. For example, a mammal that receives a “preventive” vaccine composition is less likely to acquire a particular disease or condition than a mammal that did not receive the “preventive” vaccine composition, but may nonetheless acquire the disease or condition. In certain situations, a mammal that acquires a disease or condition despite the administration of a “preventive” vaccine may nonetheless experience less virulent symptoms than a mammal that did not receive the preventive vaccine.

The terms “treatment” or “treating” include any desirable effect on the symptoms or pathology of a disease or condition, and may include even minimal reductions in one or more measurable markers of the disease or condition being treated. “Treatment” does not necessarily indicate complete eradication or cure of the disease or condition, or associated symptoms thereof. The subject receiving this treatment is any animal in need, including primates, in particular humans, and other mammals such as equines, cattle, swine and sheep; and poultry and pets in general.

In the discussion below, the nature of various immunomodulatory flagellin polypeptides and fusion proteins that comprise them are described. In many cases, these polypeptides or fusion proteins will be produced recombinantly and constructs for their production are part of the invention. Thus, the description of the polypeptides or fusion proteins applied equally to the nucleotide sequences encoding them, and vice versa. That is, description of the nature of polypeptides also includes inherent description of the nucleotide sequences encoding them and description of nucleotide sequences encoding polypeptides or proteins inherently describes the characteristics of these encoded amino acid sequences. Unless it is evident from the context, therefore, descriptions of amino acid sequences also inherently describe their encoding nucleotide sequences and vice versa.

The immunomodulatory flagellin polypeptides provided herein function generally to stimulate an innate immune response, which, as noted above, may not only enhance an adaptive immune response, but may provide beneficial immune related responses independent of the adaptive immune response. The vaccine compositions include nucleic acids that encode an immunomodulatory flagellin polypeptide and direct its expression, thereby stimulating certain aspects of the innate immune response. For example, a flagellin polypeptide present on the surface of an infected cell, bacterium, parasite, or virus particle, or released into the extracellular environment by secretion or cell lysis, may interact with and/or stimulate toll-like receptor molecules, such as toll-like receptor 5, present on the surface of certain mammalian cells, including immune cells and stromal cells. Alternatively, a flagellin polypeptide present in the cytosol of a mammalian cell, for example, during infection with a flagellin-expressing transgenic virus or bacterium, may interact with and/or stimulate an Ipaf-mediated signaling pathway within the cell. In other aspects, a flagellin polypeptide expressed within the context of a live vaccine as provided herein may interact with and/or stimulate both TLR5 and Ipaf mediated pathways, thereby providing a synergistic effect with respect to enhancing an immune response.

Innate immune cells, such as macrophages and dendritic cells, are able to determine whether flagellin remains outside the mammalian cell, or if it gains access to the cytosol and Ipaf activation by flagellin occurs independently of TLR5 activation. Not wishing to be bound by any theory, certain embodiments of the present invention provide the advantage of being able to activate both an Ipaf-mediated immune response and a TLR5-mediated immune response. In illustration, previous vaccine-related methods utilize exogenously produced, isolated, and purified flagellin polypeptides as a vaccine adjuvant. But the direct administration of isolated flagellin polypeptides generally does necessarily not allow these polypeptides to enter the cytoplasm of a targeted immune cell, at least in a functionally intact form that is capable of stimulating Ipaf. Since Ipaf is an intracellular pathway, direct administration of isolated, exogenous flagellin polypeptides to a mammal generally does not stimulate an Ipaf-mediated immune response, but instead merely interacts with and/or stimulates cell surface TLR5 molecules. In contrast, flagellin polypeptides administered according to certain embodiments of the present invention may be expressed intracellularly, such as in the cytoplasm, or may be injected into the cytoplasm by a bacterial host and, thus, may not only stimulate TLR5 molecules on the cell surface, e.g. upon release of flagellin following cell lysis, but may also stimulate the intracellular Ipaf-signaling pathway.

Intracellular production and expansion of flagellin polypeptides by single administration of live vaccine agents also provides enhanced and sustained immunomodulatory activity (i.e., immune response) as compared to a single administration of exogenously produced flagellin polypeptides. This advantage allows the use of a smaller initial vaccine dosage, or a smaller “immunogenic amount,” without the need for either repeated administration of exogenous flagellin polypeptide or reliance on large amounts of exogenous produced and purified flagellin protein.

It can readily be determined if a vaccine formulation induces an innate, humoral, cell-mediated, or any combination of these types of immune response, as methods for characterizing these immune responses are well known in the art. Detection of an innate immune response can be generally achieved within hours or days of vaccine administration. The ability of a vaccine composition or formulation to induce a humoral response can be determined by measuring the titer of antigen-specific antibodies in a mammal primed with the vaccine composition, or determining the presence of antibodies cross-reactive with an antigen by ELISA, Western blotting or other well-known methods. Cell-mediated immune responses can be determined, for example, by measuring cytotoxic T cell response to antigen using a variety of methods well known in the art.

Immunomodulatory Flagellin Polypeptides

All of the compositions of the invention contain “immunomodulatory flagellin polypeptides.” As is understood in the art, these polypeptides are portions of flagellin monomer or defined variants thereof that effect an innate immune response that comprises a TLR5-mediated immune response, an Ipaf-mediated immune response or both.

This response effected extracellularly through interaction with TLR5 employs relevant portions in the D1 domains, which includes contiguous residues from the amino and carboxy domains of the protein, further defined below. D1 forms the top of the stem and the elbow of the “gamma” shaped flagellin monomer. Compositions designed to interact with TLR5 will thus contain at least the effective portions of the D1 domain. The compositions may consist essentially of these portions of this domain or may consist of these portions of this domain. Intracellular triggering of the response results from interaction of the D0 domain with Ipaf/NLRC4. Compositions designed to interact with Ipaf will thus contain at least substantially the D0 domain, which refers to the carboxy terminal approximately 35 amino acids of the monomer. The compositions may consist essentially of this domain or may consist of this domain. The foregoing statements refer both to these domains at the polypeptide/amino acid level and to the relevant encoding nucleic acids which may be RNA or DNA depending on the specific constructs and methodology.

The immunomodulatory flagellin polypeptides may have precisely the same amino acid sequence as the relevant portion of a native flagellin monomer or may deviate in inconsequential ways from such sequences. In general, the relevant sequences are conserved among bacterial species as will be apparent from the full length flagellin sequences exemplified herein as SEQ ID NO:1-SEQ ID NO:23. Substitutions, especially substitutions of non-critical residues in these regions, may be tolerated and embodiments of these regions with such substitutions are included within the scope of the invention. This immunomodulatory function is dictated by the ability of flagellin to bind to TLR5 or to activate Ipaf.

For TLR5 activation, precise mapping of the sequences involved has been performed. The recognition site requires residues from at least two contiguous stretches of amino acids within the D1 domain of the protein: 1) residues 88-114 and 2) residues 411-431 (in Salmonella typhimurium FliC flagellin (Smith, Nature Immunology (2003) 4:1247-1253 (supra))). Of these two regions, the residues 88-100 are particularly strongly conserved and can best be considered as a signature for TLR5 activation. Within the 13 amino acids in the 88-100 region, at least 6 substitutions are permitted between Salmonella flagellin and other flagellins that still preserve TLR5 activation (such as Serratia marcescens, which has 6 substitutions), while sequences containing 8 mutations from Salmonella are not detected (such as Helicobacter pylori) (E. Andersen-Nissen, PNAS (2005) 102:9247-9252). Therefore, immunomodulatory flagellin polypeptides include flagellin like sequences that activate TLR5 and contain a 13 amino acid motif that is 53% or more identical to the Salmonella sequence in 88-100 of FliC (which is LQRVRELAVQSAN). Certain amino acids within this motif are invariant and cannot be mutated while maintaining TLR5 activation (Smith, 2003, supra). These include the residues that are underlined from this TLR5 activating motif: LQRVRELAVQSAN.

The flagellin motif that activates Ipaf is less well defined, but it lies in the carboxy terminal 35 amino acids of the flagellin protein. Within this region, there are 15 substitutions between Legionella pneumophila FlaA flagellin and Salmonella typhimurium FliC flagellin, both of which are detected through Ipaf. Thus, the immunomodulatory flagellin polypeptides include flagellin like immunomodulatory sequences that activate Ipaf and that contain a 35 amino acid sequence that is at least 57% identical to the carboxy-terminal 35 amino acids of Salmonella typhimurium FliC flagellin having the sequence

TEVSNMSRAQILQQAGTSVLAQANQVPQNVLSLLR.

In an alternative definition, variants of a native bacterial flagellin monomer sequence are included within the definition of “immunomodulatory flagellin polypeptide” as long as the immunomodulatory function is preserved and as long as the overall amino acid sequence is at least 85% or 95% (or 97% or 99%) identical to at least one native region specified. In this form of the definition of immunomodulatory flagellin polypeptide, the D0 region corresponds to the C-terminal amino acids of S. typhimurium and the D1 region is defined as that corresponding to residues 88-114 plus residues 411-431 of the S. typhimurium flagellin described by Smith (supra). Critical residues which do not tolerate substitutions have been identified in the art, in particular in published U.S. patent application 2005/0147627, referenced above, as well as described in Smith, K. D., et al., Nat. Immunol. (2003) 4:1247-1253 also cited above. Thus, the structure function relationships of the amino acid sequences in the relevant regions of the immunomodulatory flagellin polypeptides are understood in the art.

The percent identity between two amino acid or nucleotide sequences can be determined using the algorithm of E. Meyers and W. Miller (Cabios (1989) 4:11-17) which has been incorporated into the ALIGN program (version 2.0), using a PAM120 weight residue table, a gap length penalty of 12 and a gap penalty of 4.

The immunomodulatory flagellin polypeptides of the invention can also be defined in terms of the nucleotide sequences that encode them. Thus, the immunomodulatory flagellin polypeptides of the invention include those encoded by polynucleotides that hybridize under specified stringency conditions to polynucleotides that encode any of the D0 and/or D1 regions of the native flagellin proteins set forth in SEQ ID NO: 1-SEQ ID NO:23, wherein the D0 and D1 regions are defined as set forth above. Thus, the flagellin polypeptides include those encoded by polynucleotides that hybridize to these reference flagellin nucleotide sequences, or to their complements, under medium stringency or high stringency. Guidance for performing hybridization reactions can be found in Ausubel, et al., (1998, supra), Sections 6.3.1-6.3.6. Medium stringency refers to hybridizing in 6×SSC at about 45° C., followed by one or more washes in 0.2×SSC, 0.1% SDS at 60° C. High stringency conditions refer to hybridizing in 6×SSC at about 45° C., followed by one or more washes in 0.2×SSC, 0.1% SDS at 65° C.

In some embodiments, a flagellin polypeptide is encoded by a polynucleotide that hybridizes to a disclosed nucleotide sequence under “very high” stringency conditions, which refers to hybridizing 0.5 M sodium phosphate, 7% SDS at 65° C., followed by one or more washes at 0.2×SSC, 1% SDS at 65° C.

Fusion Proteins

The present invention also contemplates the use of flagellin chimeric or fusion proteins for generating an immune response in a mammal. As used herein, a flagellin “chimeric protein” or “fusion protein” includes a flagellin polypeptide linked to a non-flagellin polypeptide. A “non-flagellin polypeptide” refers to a polypeptide having an amino acid sequence corresponding to a protein which is different from the flagellin protein and which is derived from the same or a different organism. The flagellin polypeptide of the fusion protein can correspond to all or an immunomodulatory portion, e.g., a fragment described herein of an flagellin amino acid sequence. A flagellin fusion protein includes at least relevant portions of the D0 or D1 regions or both of the flagellin protein. The non-flagellin polypeptide can be fused to the N-terminus or C-terminus of the flagellin polypeptide.

The fusion protein can include a moiety or linker sequence that has a high affinity for a ligand. For example, the fusion protein can be a GST-flagellin fusion protein in which the flagellin sequences are fused to the C-terminus of the GST sequences, or one or more different epitope tags known to a person skilled in the art. A flagellin polypeptide may be fused to an epitope from GSK3b or an influenza HA epitope, or to a dual epitope tag containing both an epitope from GSK3b and the influenza HA epitope, which is recognized by monoclonal antibody HA.11, i.e., to the amino acid sequence MSGRPRTTSFAESLDYPYDVPDYA. The D2 and D3 domains of a flagellin polypeptide may be removed and replaced with a linker domain, such that the amino and carboxy terminal segments of the D1 (including if desired, D0) domains are bridged by the linker domain. The linker domain may comprise any functional heterologous polypeptide sequence. Such fusion or chimeric proteins can facilitate the identification of flagellin within the context of the vaccine composition, and can contribute to protein folding and stability.

In certain host cells, secretion of flagellin proteins can be regulated through use of a heterologous signal sequence, thus the heterologous peptide may be a signal sequence. The heterologous peptide may also enhance cell penetration; for example, a flagellin fusion polypeptide may comprise a protein transduction domain or cell-penetrating peptide, such as those described for the HIV transcription factor Tat and the Drosophila transcription factor Antennapedia, among others (see Green, et al., TRENDS in Pharmacological Sciences (2003) 24:213-215; Chauhan, et al., J Control Release (2007) 117:148-162; and Vives, et al., J Biol Chem (1997) 272:16010-16017, each of which are herein incorporated by reference). The inclusion of a protein transduction domain facilitates uptake of a flagellin polypeptide by neighboring cells upon secretion of the polypeptide from a bacterial cell or virus infected cell, including cell lysis. In certain embodiments, the cell-penetrating peptide may comprise the amino acid sequence RKKRRQR, which is derived from HIV tat.

Further, certain post-translational modifications may be used to deliver flagellin containing proteins to the cytosol of mammalian cells. The myristoyl group is a naturally occurring posttranslational modification that serves to target cytoplasmic proteins to intracellular membranes, such that myristoylation of a polypeptide leads to membrane targeting, however, myristoylation has also been shown to deliver extracellular protein to the cytosol (Nelson, et al., Biochemistry (2007) 46:14771-14781). The enzyme N-myristoyltransferase catalyzes the covalent attachment of myristate to the N-terminus of various proteins according to the presence of an appropriate sequence motif (Maurer-Stroh, et al., J Mol Biol. (2002) 317:523-540). Accordingly, the present invention contemplates flagellin polypeptides modified with an appropriate protein myristoylation motif to allow the attachment of a myristoyl group to the N-terminus of the flagellin polypeptide during production in vitro. Subsequent delivery of this protein to an animal will result in the delivery of flagellin to the cytosol and activation of Ipaf.

In important embodiments, the heterologous amino acid sequence fused to the immunomodulatory flagellin polypeptide will be one or more antigens for which an adaptive immune response is desired. Such antigens include antigens representative of infectious agents, including viruses, bacteria and parasites; antigens that represent endogenous targets, such as tumor-associated antigens; and any other sequence to which an immune response is desired. Suitable viral and bacterial antigens are associated with the diseases against which the vaccines may be targeted as described in detail below. The nature of tumor-associated antigens is also well known in the art, and such antigens are often based on individual expression in endogenous tumors.

Viral Vaccines

In one aspect of the invention the compositions comprise an isolated, replication-competent or infectious virus that encodes and expresses an immunomodulatory flagellin polypeptide upon entering and infecting a target cell. The replication-competent virus may be attenuated, such that it replicates within a host but does not cause a significantly pathological condition. It is believed that the endogenous expression of a flagellin polypeptide within the cytoplasm of a virally infected cell offers advantages over the use of exogenously added flagellin as part of a vaccine, such as when using flagellin polypeptides as an adjuvant for a viral vaccine. For example, endogenous expression of immunomodulatory flagellin polypeptides within a virally infected cell allows for stimulation of the intracellular Ipaf-signaling pathways, which stimulate the innate immunity in a manner distinct from the stimulation of cell-surface TLR5.

Viral expression of the flagellin polypeptide may release the polypeptide into the cytosol of infected cells, thus activating Ipaf and may also do so when the flagellin protein is fused to a viral surface protein. These viruses will activate TLR5 as well. Viruses expressing the flagellin as a cytosolic protein also will activate TLR5 when the virally infected cell lyses.

In certain embodiments, the replication-competent virus is selected from Adenoviridae, Caliciviridae, Picornoviridae, Herpesviridae, Hepadnaviridae, Filoviridae, Flaviviridae, Retroviridae, Orthomyxoviridae, Papovaviridae, Parvoviridae, Poxviridae, Reoviridae, Togaviridae, and Influenzae. The virus may express the immunomodulatory flagellin polypeptide within an infected cell.

Thus, examples of viruses that may be used to construct a vaccine as presently claimed include, but are not limited to, members of the Adenoviridae family, including human adenoviruses A through F; members of the Caliciviridae family, such as Norwalk virus (or norovirus); members of the Picornoviridae family, including, for example, enteroviruses A through D, poliovirus, rhinoviruses A and B, Hepatitis A virus, encephalomyocarditis virus, foot and mouth disease, human perchoviruses 1 through 6, equine rhinitis B viruses 1 to 3; and members of the Herpesviridae family, including, for example, human herpes viruses 1 through 8 (HHV1-8), also known as herpes simplex virus (HSV)-1, HSV2, varicella zoster virus, Epstein-Barr virus, cytomegalovirus, roseolovirus, Kaposi's sarcoma-associated herpesvirus (KSHV). Additional examples of Herpesviridae include bovine herpesviruses, equine herpesviruses, canine herpesviruses, and feline herpesviruses.

Additional examples of Hepadnaviridae, include Hepatitis B virus; Filoviridae, including, for example, hemorrhagic fever viruses such as Ebola viruses and Marburg viruses; and Flaviviridae, including, for example, dengue fever viruses, Japanese encephalitis viruses, Murray Valley encephalitis viruses, St. Louis encephalitis viruses, Tick-born encephalitis viruses, West Nile viruses, yellow fever viruses, and hepatitis C virus. Examples of Retroviridae that may be utilized according to the present invention include, for example, alpharetroviruses such as Rous sarcoma virus, UR2 sarcoma virus, and Y73 sarcoma virus; betaretroviruses such as mouse mammary tumor virus, Jaagsiekte sheep retrovirus, Mason-Pfizer monkey virus, and Langur virus; gammaretroviruses such as murine leukemia viruses, feline leukemia viruses, Gibbon ape leukemia viruses, feline sarcoma viruses, and murine sarcoma viruses; deltaretroviruses such as bovine leukemia virus, primate T-lymphotropic virus and human T-lymphotropic virus; lentiviruses such as human immunodeficiency virus (HIV)-1, HIV-2, simian immunodeficiency viruses, bovine immunodeficiency viruses, equine immunodeficiency viruses, feline immunodeficiency viruses, and Visna/maedi viruses; and spumaviruses such as macaque foamy viruses, bovine foamy viruses, equine foamy viruses, feline foamy viruses, and human foamy viruses.

Additional examples of viruses include Orthomyxoviridae, such as influenzaviruses A through C; Paramyxoviridae, such as measles viruses, mumps viruses, sendai virus, parainfluenza viruses 1 and 3, human and bovine respiratory syncytial viruses, human metapneumoviruses, Rinderpest virus, and canine distemper virus, Papovaviridae, including, for example, papillomaviruses, such as human papillomavirus (HPV)-1, HPV-2, HPV-4, HPV-3, HPV-5, HPV-6, HPV-7, HPV-10, HPV-11, HPV-13, HPV-16, and HPV-18, HPV-31, HPV-32, HPV-33, HPV-35, HPV-39, HPV-42, HPV-43, HPV-44, HPV-45, HPV-51, HPV-55, among others, and polyomaviruses, such as SV40; and Parvoviridae, such as B19 virus, adeno-associated viruses (AAV)-1, AAV-2, AAV-5, AAV-6, AAV-7, AAV-8, among others, including hybrids thereof. Additional examples include Poxviridae, such as vaccinia virus, cowpox, smallpox, molluscum contagiosum virus; Reoviridae, such as mammalian orthoreoviruses, rotavirus A, Colorado tick fever virus; and Togaviridae, such as Sindbis virus, Eastern equine encephalitis virus, Western equine encephalitis virus, Venezuelan equine encephalitis virus, Ross River virus, O'nyong'nyong virus, and Rubella viruses.

Influenza is a serious and common viral infection for which improved vaccines would be important. Therefore, the examples below highlight the utility of the flagellin polypeptides in influenza vaccines as an example of insertion into a viral vaccine vector. The viral vaccine may comprise an influenza virus (IV), such as an influenzavirus A, influenzavirus B, or an influenzavirus C, wherein an immunomodulatory flagellin polypeptide is inserted into the genome of the influenza virus. An IV is roughly spherical, but it can also be elongated or irregularly shaped. Inside the virus, eight segments of single-stranded RNA contain the genetic instructions for making the virus. The most striking feature of the virus is a layer of spikes projecting outward over its surface. There are two different types of spikes: one is composed of the molecule hemagglutinin (HA), the other of neuraminidase (NA). The HA molecule allows the virus to “stick” to a cell, initiating infection. The NA molecule allows newly formed viruses to exit their host cell without sticking to the cell surface or to each other. The viral capsid is comprised of viral ribonucleic acid and several so called “internal” proteins (polymerases (PB1, PB2, and PA, matrix protein (M1) and nucleoprotein (NP)). Because antibodies against HA and NA have traditionally proved the most effective in fighting infection, much research has focused on the structure, function, and genetic variation of those molecules. Influenza viruses also contain two non-structural proteins M2 and NS1, both of which play important roles in viral infection.

Influenza virions contain 7 segments (influenza C virus) or 8 segments (influenza A and B virus) of linear negative-sense single stranded RNA. Most of the segments of the virus genome code for a single protein. For many influenza viruses, the whole genome is now known. Genetic reassortment of the virus results from intermixing of the parental gene segments in the progeny of the viruses when a cell is co-infected by two different viruses of a given type. This phenomenon is facilitated by the segmental nature of the genome of influenza virus. Genetic reassortment is manifested as sudden changes in the viral surface antigens.

The flagellin polypeptide may inserted into an influenza coding region, such as a nucleotide sequence that encodes a viral polypeptide, or may be inserted into the genome without interfering with the coding region of a viral polypeptide. The flagellin polypeptide may be operably linked to an influenza viral promoter, or to a heterologous promoter, such as a CMV promoter, ubiquitin promoter, or other promoter known in the molecular biological arts.

The influenza virus may be attenuated, for example, by creating one or more deletions and/or mutations in a virulence gene, such as a gene associated with the pathogenicity of an influenza infection. In one example, an influenza virus may be attenuated by altering the wild-type NS-1 gene, which otherwise contributes to the pathogenicity of the influenza virus by inserting the coding sequence for the flagellin polypeptide into the coding sequence of the NS-1, or the virus may encode the flagellin polypeptide fused to either the N-terminus or C-terminus of an complete or partial NS-1 nucleotide sequence. The NS1 gene may be truncated by the addition of a start/stop sequence, downstream of which contains a coding sequence for a flagellin polypeptide, by a start/stop sequence at amino acid 125 by a start/stop sequence (TAATG), which stops NS1 after amino acid 125 and provides a start codon for a flagellin polypeptide coding sequence.

A polynucleotide that encodes a flagellin polypeptide may be inserted into other viral polypeptide coding sequences, for example, into the NA, HA and/or M protein coding sequences, which are localized to the surface of the influenza virus particle. Not wishing to be bound by any theory, it is believed that flagellin polypeptide expression on the viral surface could stimulate various TLR5-mediated cellular responses upon virus interaction with the cell, while subsequent intracellular expression of a flagellin polypeptide by the infected cell would stimulate Ipaf-mediated cellular responses, thereby providing a synergistic enhancement of the immune response to the viral vaccine.

WO94/21797, incorporated herein by reference in its entirety, discloses IV vaccine compositions comprising DNA constructs encoding NP, HA, M1, PB1 and NS1, and also discloses methods of protecting against IV infection comprising immunization with a prophylactically effective amount of these DNA vaccine compositions.

The present invention also contemplates the use of various viral vectors or nucleic acid constructs to generate an enhanced immune response to one or more desired polypeptide antigens. A viral vector or construct may comprise a polynucleotide sequence that encodes a flagellin polypeptide in addition to a polynucleotide sequence that encodes a desired polypeptide antigen, such as a viral antigen, a tumor antigen, a bacterial antigen, and/or a parasitic antigen. The viral vector employed may or may not be related to the desired antigen. For example, a retroviral (e.g., MLV or lentiviral vector), vaccinia, herpes, or adeno-associated viral vector may be employed to deliver a tumor or bacterial antigen, or an antigen from an unrelated virus. Examples of viral vectors include adenovirus, canarypox, vesicular stomatitis virus, adeno-associated virus, poxvirus, alphavirus replicon, and replicating adenovirus 4.

A viral vector may be replication-competent upon administration to a mammal, as described herein, or it may be competent only for a single round of infection upon administration. Typically, the polynucleotide sequences encoding the flagellin polypeptide and the desired antigen are operably linked to one or more promoter sequences. In certain embodiments, the flagellin polypeptide and the desired polypeptide antigen may form a fusion or chimeric protein. As such, a viral vector delivery system comprising an endogenously expressed flagellin polypeptide may be utilized to generate an enhanced immune response to any desired antigen.

Bacterial Vaccines

The present invention also includes the use of live attenuated bacterial vaccines, wherein the bacteria comprises an exogenous nucleotide sequence that encodes an immunomodulatory flagellin polypeptide, and wherein the exogenous nucleotide sequence is operably linked to a bacterial promoter.

Depending on the nature of the bacterial strain, the encoding sequence for the immunomodulatory peptide may need to be provided with an operably linked sequence encoding a signal sequence. Secretion signals are well known in the art, and if TLR5 activation is to be effected, a signal sequence should be provided. However, if the bacteria strain express flagellin that evades TLR5 (for example, Helicobacter and Campylobacter), a heterologous flagellin polypeptide that does not evade TLR5 could be expressed and secreted through the native flagellar secretion apparatus to activate TLR5 from the extracellular space. These bacteria would not, without further modification, be expected to activate Ipaf.

If the infection of the bacterial strain results in an escape of the bacteria into the cytosol, as is demonstrated for Shigella, Listeria, as well as others, then a secretion signal may be added to export the protein outside the bacteria into the cytosol so as to activate Ipaf. TLR5 will also be activated upon lysis of the infected cell or when the bacteria resides outside or between host cells. Thus, the skilled artisan will understand how to provide for appropriate secretion of the polypeptide to provide adequate access to TLR5 or Ipaf or both. Thus, the modified bacteria will elicit both an innate response due to the interaction of the flagellin polypeptide with the appropriate receptor as well as an enhanced adaptive response to the antigens present on the bacteria.

If the bacterial strain used in the infection expresses certain virulence factors secretion apparatuses, then these may facilitate the transport of flagellin into the cytosol of host cells, thereby activating Ipaf. Examples include the type III secretion system (such as found in Salmonella) and the type IV secretion system (such as found in Legionella). Flagellin can be translocated from the bacterial cytosol to the host cytosol by these two systems without the addition of heterologous secretion signals, however the flagellar charperone protein (FliS in Salmonella typhimurium) may be required. Thus, for a bacteria that expresses a type III secretion system but does not express flagellin, flagellin can be expressed in the bacteria, resulting in translocation of flagellin into the host cytosol that is detected by Ipaf. Example bacteria for which this may be useful are Salmonella spp and Yersinia pestis.

Live bacterial vaccines include bacterial strains that replicate in a host, so that the vaccine may elicit an immune response similar to that elicited by the natural infection. A live bacterial vaccine may be attenuated, meaning that its disease-causing capacity is minimized or eliminated by biological or technical manipulations. Typically, a live bacterial vaccine is neither underattenuated, i.e., retaining even limited pathogenicity, nor overattenuated, i.e., being no longer infections enough to be an effective vaccine. Live bacterial vaccines usually elicit both humoral immunity as well as cellular immunity. Live bacterial vaccines containing an exogenous flagellin polypeptide, described herein, are predicted to elicit increased innate immune responses that will promote more vigorous humoral and cellular immune responses in turn.

Live attenuated bacterial vaccines may be produced by classical strategies, such as in vitro culturing under conditions to suppress virulence factors. For example, a tuberculosis vaccine consists of a live attenuated strain of Mycobacterium bovis (BCG vaccine), which was attenuated by successive in vitro subculturing methods, and has been inoculated into billions of people worldwide. The BCG vaccine, however, varies in immunogenicity and in the rate of protective efficacy in clinical trials. Certain embodiments of the present invention may include a live attenuated BCG vaccine that comprises an exogenous flagellin-encoding polynucleotide sequence, as described herein, to enhance the immunogenicity of this vaccine.

Live attenuated bacterial vaccines may also be produced by chemical mutagenesis. For example, Ty21a strain of Salmonella typhi was derived according to chemical mutagenesis techniques, and is licensed for preventing or reducing the risk of typhoid fever. The present invention, thus, contemplates the use of attenuated vaccines produced by chemical mutagenesis, such as that Ty21a strain of Salmonella, wherein the chemically mutated bacteria comprises an exogenously provided flagellin polypeptide-encoding sequence to enhance the immunogenicity of this vaccine agent, such as by stimulating TLR5 and/or Ipaf-mediated cellular immune responses. Thus, for example, Ty21a Salmonella that express flagellin from a constitutive promoter could elicit greater immune responses than the parental Ty21a strain.

Live attenuated bacterial vaccines may also be produced by recombinant techniques. For example, one strategy may involve the identification of genes responsible for virulence, colonization, and/or survival and to either eliminate the gene or genes or to abolish or modulate the in vivo expression of such genes. In certain embodiments, it may be desirable to delete two or more independent genes or genetic loci that contribute to virulence, to reduce the possibility of reversion. For example, a licensed Vibrio cholerae vaccine is based on a strain produced by deleting genes that encode virulence factors (e.g., cholera toxin). In addition, Shigella strains have been developed by mutating particular plasmid or chromosomal genes to reduce pathogenicity. As such, the present invention contemplates the use of bacterial vaccines attenuated by recombinant techniques, such as the Vibrio and Shigella vaccines, as known in the art, wherein the bacteria contains an exogenous polynucleotide sequence that encodes an immunomodulatory flagellin polypeptide, as provided herein.

Live attenuated bacterial strains that comprise an exogenous nucleotide sequence that encodes an immunomodulatory flagellin polypeptide wherein the exogenous nucleotide sequence is operably linked to a bacterial promoter are part of the invention. The bacterial strain may be one that does not contain an endogenous flagellin gene, such as bacteria selected from Mycobacterium tuberculosis, Mycobacterium leprae, Yersinia pestis, Neisseria gonorrhea, Chlamydia trachomatis, Chlamydia pneumoniae, Streptococcus pneumoniae, Staphylococcus aureus, group A Streptococcus, group B Streptococcus, Neisseria meningiditis, Haemophilus influenzae, and Acinetobacter baumii.

In other embodiments, the bacteria comprises an endogenous flagellin polypeptide that does not induce an TLR5 mediated-immune response or an Ipaf-mediated immune response. Such bacteria include, for example, Helicobacter pylori and Camphylobacter jejuni.

In other embodiments, the bacteria is modified so as not to produce endogenous flagellin polypeptide sequence that is capable of inducing a TLR5 and/or an Ipaf-mediated response. Such bacteria may be selected from Salmonella typhimurium, Salmonella typhi, Salmonella paratyphi, Salmonella enteriditis, and Listeria monocytogenes.

In certain embodiments, a bacteria comprising an exogenously provided flagellin polynucleotide sequence may express a flagellin polypeptide as a surface component of the bacteria, or as a secreted molecule. In certain embodiments, the bacteria is of the type that is capable of replicating within a mammalian host cell (i.e., intracellular replication). A bacterial vaccine may comprise a bacteria that contains an endogenous flagellin-encoding nucleotide sequence, or it may not contain such an endogenous sequence. For example, a bacterial vaccine may comprise a non-flagellated bacteria, a flagellated bacteria that does not naturally induce a TLR5 or Ipaf-mediated cellular response, and/or a flagellated bacteria that contains a flagellin polypeptide that is capable of inducing a TLR5 or Ipaf mediated cellular response, but nonetheless suppress endogenous flagellin expression to avoid activating the innate immunity of the infected host.

Examples of non-flagellated bacteria include (i.e., bacteria that typically do not contain an endogenous flagellin gene), but are not limited to, Mycobacterium tuberculosis, Mycobacterium leprae, Yersinia pestis, Neisseria gonorrhea, Chlamydia trachomatis, Chlamydia pneumoniae, Streptococcus pneumoniae, Staphylococcus aureus, group A Streptococcus (GAS), group B Streptococcus (GBS), Neisseria meningiditis, Haemophilus influenzae, and Acinetobacter baumii. Without wishing to be bound by any theory, it is believed that incorporating a polynucleotide sequence that expresses a flagellin polypeptide as a cell surface or secreted molecule of an otherwise non-flagellated bacterium will stimulate a TLR5 and/or Ipaf-mediated cellular response, thereby enhancing the innate and adaptive immune response to a given non-flagellated bacterial vaccine.

Examples of flagellated bacteria that contain an endogenous flagellin gene but do not induce a TLR5-mediated or Ipaf-mediated immune response include (i.e., TLR5 and/or Ipaf fail to interact with endogenous bacterial flagellin protein), but are not limited to, Campylobacter jejuni and Helicobacter pylori. In illustration, it has been shown that despite the fact that TLR5 recognizes a highly conserved domain in flagellin, some flagellated bacteria contain sequence changes in the D1 domain that prevent detection by TLR5. For example, as noted above, the r-Proteobacteria, including the human pathogens Campylobacter jejuni and Helicobacter pylori, contain sequence changes that permit TLR5 evasion as well as compensatory mutations that restore flagellin polymerization and motility. It is believed that the addition of an exogenous polynucleotide sequence, which encodes and expresses an immunomodulatory flagellin polypeptide as described herein, would stimulate TLR and/or Ipaf-mediated cellular responses, and thereby enhance the immune response to these types of bacteria.

Examples of flagellated bacteria that contain a flagellin polypeptide that is capable of inducing a TLR5 or Ipaf mediated response, but otherwise suppress the expression of said flagellin gene to avoid activating innate immunity include, but are not limited to, Salmonella typhimurium, Salmonella typhi, Salmonella paratyphi, Salmonella enteriditis, and Listeria monocytogenes. For these type of bacteria, it is believed that placing an immunomodulatory flagellin polypeptide under the control of a promoter, such as a bacterial promoter, which cannot be suppressed by the bacteria would stimulate TLR and/or Ipaf-mediated cellular responses, and thereby enhance the immune response to these types of bacteria.

An exogenous polynucleotide sequence that encodes an immunomodulatory flagellin polypeptide may be introduced into a bacteria using known techniques in the art.

Eukaryotic Parasitic Organism Vaccines

The present invention also contemplates the use of a vaccine composition comprising a eukaryotic parasitic organism, wherein the parasitic organism comprises an exogenous nucleotide sequence that encodes an immunomodulatory flagellin polypeptide, and wherein the exogenous nucleotide sequence is operably linked to a promoter. Examples of parasitic organisms include, but are not limited to, Entemoeba histolytica, Necator americanus, Ancylostoma duodenale, Leishmania, Plasmodium falciparum, P. vivax, P. ovale, P. malariae), Schistosoma mansoni, S. haematobium, S. japonicum, Onchocerca volvulus, Trypanosoma cruzi, and Dracunculus medinensis.

Compositions/Formulations for Administration

The present invention encompasses pharmaceutical compositions and vaccine compositions comprising the replication-competent viruses, viral vectors, live attenuated bacteria, and/or eukaryotic parasitic organisms described herein (i.e., vaccine agents) or fusion proteins. Pharmaceutical compositions typically comprise a pharmaceutically acceptable carrier or excipient in combination with the vaccine agents of the present invention. Vaccine compositions typically comprise an additional pharmaceutically acceptable adjuvant in combination with the vaccine agents of the present invention.

The pharmaceutical and vaccine compositions of the present invention may be administered according to any appropriate route of administration, including, but not limited to, inhalation, intradermal, transdermal, intramuscular, topically, intranasal, subcutaneous, direct injection, and formulation.

Compositions of the present invention may include solutions of the active vaccine agents as provided herein (e.g., viruses or bacteria), which may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of undesirable microorganisms. The pharmaceutical forms suitable for injectable use include sterile aqueous solutions or dispersions and sterile powders for the extemporaneous preparation of sterile injectable solutions or dispersions In all cases the solution form should be sterile and fluid to the extent that easy syringability exists. It should be stable under the conditions of manufacture and storage and should be preserved against the contaminating action of unwanted microorganisms, such as bacteria and fungi unrelated to the vaccine agent provided therein. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. In certain embodiments, in which live bacteria are not included in the composition, the prevention of the action of undesired microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In general, suitable formulations are described in Remington's Pharmaceutical Sciences, latest edition, Mack Publishing Co., Easton, Pa., incorporated herein by reference.

Methods of Treatment/Diseases

The present invention contemplates utilizing the vaccines provided herein for treating or reducing the risk of acquiring a wide variety of disease or conditions, including infectious diseases such as viral infections, bacterial infections, and parasitic infections, in addition to conditions caused by pathologically aberrant cells, such as degenerative conditions or cancer.

Examples of viral infectious diseases or agents include, but are not limited to, Hepatitis A, Hepatitis B, Hepatitis C, Hepatitis E, Caliciviruses associated diarrhea, Rotavirus diarrhea, Haemophilus influenzae B pneumonia and invasive disease, Influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, HIV/AIDS, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, leprosy, upper and lower respiratory infections, poliomyelitis, among others described elsewhere herein.

Examples of bacterial infections disease or agents include, but are not limited to, Bacillus antracis, Borellia burgdorferi, Brucella abortus, Brucella canus, Brucella melitensis, Brucella suis, Campylobacter jejuni, Chlamydia pneumoniae, Chlamydia psitacci, Chlamydia trachomatis, Clostridium botulinum, C. difficile, C. perfringens, C. tetani, Corynebacterium diphtheriae (i.e., diphtheria), Enterococcus, Escherichia coli, Haemophilus influenza, Helicobacter pylori, Legionella pneumophila, Leptospira, Listeria monocytogenes, Mycobacterium leprae, M. tuberculosis, Mycoplasma pneumoniae, Neisseria gonorrhea, N. meningitidis, Pseudomonas aeruginosa, Rickettsia recketisii, Salmonella typhi, S. typhimurium, Shigella sonnei, Staphylococcus aureus, S. epidermidis, S. saprophyticus, Streptococcus agalactiae, S. pneumoniae, S. pyogenes, Treponema pallidum, Vibrio cholera, Yersinia pestis, Bordatella pertussis, and otitis media (e.g., often caused by Streptococcus pneumoniae, Haemophilus influenzae, or Moraxella catarrhalis), among others described elsewhere herein.

Certain embodiments contemplate methods of treating or reducing the risk of a pathogenic parasitic infection or parasitic disease in a mammal, comprising administering to the mammal a composition comprising an isolated eukaryotic parasitic organism, wherein the parasitic organism comprises an exogenous nucleotide sequence that encodes an immunomodulatory flagellin peptide, and wherein the exogenous nucleotide sequence is operably linked to a promoter. Examples of parasitic infectious diseases include, but are not limited to, Amebiasis (e.g., Entemoeba histolytica), Hookworm Disease (e.g., nematode parasites such as Necator americanus and Ancylostoma duodenale), Leishmaniasis, Malaria (four species of the protozoan parasite Plasmodium; P. falciparum, P. vivax, P. ovale, and P. malariae), Schistosomiasis (parasitic Schistosoma; S. mansoni, S. haematobium, and S. japonicum), Onchocerca volvulus (River blindness), Trypanosoma cruzi (Chagas disease/American sleeping sickness), and Dracunculus medinensis, lymphatic filariasis.

The methods provided herein may also be used to treat or reduce the risks associated with conditions characterized by “pathologically aberrant cells,” such as cancer or degenerative conditions. For example, certain embodiments contemplate methods of treating a cancerous or degenerative condition (i.e., a condition characterized by “pathologically aberrant cells), comprising administering to the mammal a composition comprising an isolated replication-competent virus or a replication-incompetent virus (i.e., competent for a single round of infection only), wherein the replication-competent virus comprises a nucleotide sequence that encodes an immunomodulatory flagellin polypeptide, and wherein the virus comprises a nucleotide sequence that encodes a desired antigen. In certain embodiments, the desired antigen is associated with a cancer cell, such as a tumor cell, but is not significantly associated with a normal cell. For example, the cancer or tumor cell may express a characteristic antigen on its cell surface, which could provide a target for immunotherapy using a vaccine as provided herein. For example, 5T4 antigen expression is widespread in malignant tumors throughout their development, and is found in tumors such as colorectal, ovarian, and gastric tumors. 5T4 expression is used as a prognostic aid in these cases, since it has very limited expression in normal tissue, and, therefore, represents a desired antigen for use with the methods provided herein. It is believed that stimulating an enhanced immune response against an antigen associated with a cancer cell, such as by stimulating TLR5-mediated response and/or an Ipaf-mediated cellular response, will induce an immune response, such as an cellular immune response, against the cancer or tumor cell, thereby helping to destroy the cancer or tumor cell.

Examples of cancers or tumors that may be treated according to the present invention include, but are not limited to, prostate cancer, lung cancer, colorectal cancer, bladder cancer, cutaneous melanoma, pancreatic cancer, leukemia, breast cancer, endometrial cancer, non-Hodgkin's lymphoma, ovarian cancer, malignant melanoma, renal cell carcinoma, thyroid cancer, skin cancer (nonmelanoma).

The present invention also contemplates the use of the vaccine compositions provided herein as an adjunct to chemotherapy, including use with anticancer agents such as biological agents (biotherapy), chemotherapy agents, and radiotherapy agents.

Examples of radiotherapy that have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes.

The following examples are offered to illustrate but not to limit the invention.

### Example 1

### Cloning of Influenza NS with Flagellin Inserted

Flagellin was PCR amplified and inserted into the influenza NS segment by strand overlap exchange PCR. The resulting product contains the NS segment of PR8 from plasmid pHW198-NS with flagellin inserted into the NS1 gene. NS1 is truncated at amino acid 125 by a start/stop sequence (TAATG) which stops NS1 after amino acid 125 and starts the flagellin insert. We utilized the minimal flagellin sequence that would be recognized by TLR5 and Ipaf. This flagellin has the variable domain removed and contains Salmonella typhimurium flagellin fliC amino acids 1-184, an epitope tag linker (GSK-HA), followed by flagellin residues 395-494. This construct preserves the NS2 gene splice sites, which should leave NS2 uninterrupted. The PCR product was cut with ApaI and NheI and inserted in to pHW198-NS cut with the same enzymes.

### Example 2

### In Vitro Activation of TLR5 by Recombinant Influenza Virus

In this example, recombinant influenza viruses expressing flagellin are verified to activate TLR5. Two types of recombinant influenza expressing flagellin are used: 1) immunomodulatory flagellin polypeptide fused to one of the influenza envelope proteins (HA or NA), or 2) such peptide that is expressed free within the cytosol of infected cells and that can escape to the extracellular space upon rupture of the infected cell. Supernatants from cells infected with such recombinant flagellin polypeptide-expressing influenza are incubated with Chinese hamster ovary cells (CHO) that express human TLR5 along with luciferase driven by an NF-κB responsive promoter. This cell line permits evaluation of TLR5 engagement by luciferase activity. Positive luciferase production is taken as evidence for successful flagellin polypeptide expression from the recombinant virus. The flagellin polypeptide encoded by these viruses must contain the relevant portions of the D1 domain, but could contain more of the protein.

For the assay, CHO K1 cells are transfected with human or mouse TLR5 cDNA cloned into the pEF6/V5-His TOPO vector (Invitrogen), ELAM-LUC49 and pRL-TK (Promega) plasmids, selected with blasticidin, and cloned by limiting dilution. Stable clones are stimulated for 4-5 h and assayed for luciferase activity. All assays are done in triplicate, and each experiment is repeated at least three times. ‘Fold induction’ is calculated by dividing the luciferase values for the test conditions by the relative luciferase value for the control condition.

### Example 3

### In Vitro Activation of Ipaf by Recombinant Influenza Virus

In this example, recombinant flagellin polypeptide-expressing influenza are analyzed for Ipaf activation. Flagellin polypeptide is expressed as a free protein in the cytosol of infected cells. To determine Ipaf activation, macrophages are infected with the recombinant viral particles and IL-1β secretion measured in comparison to wild type influenza virus. The dependence upon Ipaf signaling is determined via the use of macrophages derived from mice lacking Ipaf, or by knock-down using shRNA or siRNA in human monocytes derived macrophages. The flagellin expressed in these viruses must contain the D0 domain, but could contain more of the protein.

### Example 4

### In Vivo Activation of TLR5 and Ipaf Signaling by Recombinant Influenza

Once the above recombinant influenza viruses have been validated by the Examples 2 and 3 above, they are used to infect mice. Mice are infected intranasally with recombinant influenza expressing flagellin polypeptide. Effects of flagellin polypeptide on viral replication, cytokine expression, histopathology, and generation of a protective adaptive immune response are compared to wild type influenza virus. These experiments are repeated in mice deficient for TLR5, Ipaf, or both, in order to determine the mechanism of action.

### Example 5

### Detection of Cytoplasmic Flagellin by Ipaf

Cytoplasmic flagellin stimulates IL-1β secretion. (a) ELISA for IL-1β production from LPS stimulated BMM treated with Ovalbumin (OVA) or various amounts of flagellin (FliC) protein. (b) ELISA for IL-1β production from BMM treated with 30 ng of flagellin (FliC) or other bacterial virulence factors that access the cytosol of macrophages during normal infection, SspH1 (Salmonella SPI1 TTSS effector) SseI (Salmonella SPI2 TTSS effector), ActA (Listeria virulence factor) or phosphate buffered saline (PBS). (c) ELISA for IL-1β production from BMM treated with 125 ng OVA or flagellin (FliC) or with protein after overnight digestion with Proteinase K. Omission of the Profect reagent is presented as a control. (d) Immunoblot for mature IL-1β secreted by BMM transfected with 60 ng of flagellin (FliC) or OVA. Cytotoxicity was negligible and equal between samples (<5%).

Ipaf is required for the response to cytoplasmic flagellin. (a-b) BMM derived from WT, Ipaf-KO or TLR5-KO mice were stimulated with LPS for 2 hours before Profect transfection with 60 ng purified Flagellin. (a) ELISA for IL-1β secretion. Differences observed between WT and TLR5-KO null BMM were not statistically significant (p>0.05) while Ipaf-KO BMM were significantly lower than WT and TLR5-KO BMM (p<0.05). (b) Processed Caspase 1 was detected by immunoblot after Profect transfection of purified flagellin or OVA for 2 hours. (c) ELISA for IL-1β secretion from WT or Ipf-KO BMM were stimulated for 24 hours with LPS (50 ng/ml) or Poly I:C (5 μg/ml) with R848 (5 μg/ml). (d) ELISA for IL-1β secretion from BMM from WT, Ipaf-KO or ASC-KO mice stimulated with 10 ng/ml LPS for 2 hours before Profect transfection with 30 ng purified Flagellin. Differences observed between WT and ASC-KO BMM were statistically significant (p<0.05). Cytotoxicity was negligible and equal between samples (<5%).

### Example 6

### Ipaf Restricts Flagellin Expressing Pathogens

S. typhimurium activates Ipaf by delivery of flagellin through the SPI1 T3SS in vitro, but during systemic infection represses expression of flagellin via the PhoP/PhoQ regulatory system. Flagellin expression is undetectable in the spleens of S. typhimurium infected mice, and Ipaf null mice do not have significantly increased susceptibility to S. typhimurium infection. In contrast, Legionella pneumophila has not adopted this evasion strategy, and maintains flagellin expression during infection, resulting in Ipaf mediated clearance.

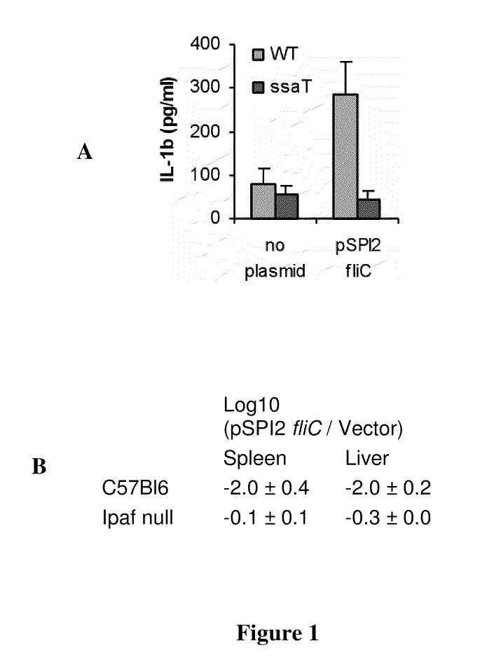

In order to study the importance of Ipaf mediated defense against flagellated pathogens, we created a strain that is unable to repress flagellin in vivo and these bacteria specifically deliver flagellin into the cytosol of host cells. We expressed FliC from a SPI2 co-regulated promoter carried on the highly stable pWSK29 expression vector (pSPI2 fliC). These bacteria secrete flagellin into the host cytosol via the SPI2 T3SS, and induce Ipaf dependent IL-1β secretion. Bone marrow-derived macrophage (BMDM) were infected with WT or SPI2 mutant (ssaT) S. typhimurium expressing pSPI2 fliC at MOI 12 for 1 hour followed by 7 hours gentamicin treatment. IL-1β secretion was determined by ELISA. The results are shown in FIG. 1A.

We are cognizant of the fact that this strain is not reflective of normal S. typhimurium pathogenesis, as WT S. typhimurium has evolved a strategy to evade Ipaf. Our intent is to use this strain as a specific probe to investigate the role of Ipaf in the innate immune response.

To test the effectiveness of this strain as a vaccine a preliminary experiment was performed. One mouse was infected with S. typhimurium expressing pSPI2 fliC orally. Two weeks later, the mouse was challenged with a lethal dose of wild type S. typhimurium. While control mice died within 5-7 days, the vaccinated mouse survived the infection with no apparent symptoms.

To examine the role of Ipaf in vivo, WT or Ipaf null mice were co-infected intraperitoneally with 5×10⁴ cfu each of S. typhimurium WT marked with kanamycin resistance, or S. typhimurium pSPI2 fliC marked with ampicillin resistance, and bacterial persistence in the spleen and liver was determined. WT or Ipaf null mice were co-infected with S. typhimurium carrying pSPI2 fliC (in pWSK29; ampicillin) or empty pWSK129 (kanamycin) vector at a 1:1 ratio. After 2 days, mice were euthanized and bacterial counts from the spleen and liver were determined. The log₁₀(pSPI2 fliC/vector) are indicated (−2 corresponds to a 100 fold decrease). The results are shown in FIG. 1B. Bacteria expressing pSPI2 fliC were defective for replication in WT mice, with 100-fold fewer bacteria recovered compared to WT S. typhimurium. This restriction was not observed in Ipaf null animals, indicating that Ipaf activation restricts bacterial growth. In these in vitro and in vivo experiments, the bacteria contain intact SPI1 and flagellin genes. However, the bacteria are grown such that SPI1 T3SS and flagellin genes are not transcriptionally active (overnight stationary phase bacterial cultures).

1. A composition for eliciting an innate immune response in a subject which composition comprises an active ingredient selected from the group consisting of
(a) an isolated replication-competent virus which comprises an expression system for a nucleotide sequence that encodes an immunomodulatory flagellin polypeptide;
(b) an isolated bacterial strain that has been modified to contain an expression system for an exogenous immunomodulatory flagellin polypeptide;
(c) a eukaryotic parasitic microorganism which comprises an expression system for an immunomodulatory flagellin polypeptide; and
(d) a fusion protein consisting essentially of an immunomodulatory flagellin polypeptide fused to an antigen and/or an amino acid sequence that facilitates cell penetration in the cells of said subject.

2. The composition of claim 1 wherein the, wherein the replication-competent virus is selected from Adenoviridae, Caliciviridae, Picornoviridae, Herpesviridae, Hepadnaviridae, Filoviridae, Flaviviridae, Retroviridae, Orthomyxoviridae, Papovaviridae, Parvoviridae, Poxviridae, Reoviridae, Togaviridae, and Influenzae.

3. The composition of claim 1 wherein, in (a) the nucleotide sequence that encodes the immunomodulatory flagellin is inserted into a nucleotide sequence that encodes a viral polypeptide.

4. The composition of claim 3 wherein the viral polypeptide is a surface protein.

5. The composition of claim 1 wherein the bacterial strain in (b) does not comprise an endogenous flagellin gene.

6. The composition of claim 1 wherein in (b) the immunomodulatory flagellin polypeptide is operably linked to a signal sequence.

7. The composition of claim 1 wherein the bacterial strain in (b) is selected from Mycobacterium tuberculosis, Mycobacterium leprae, Yersinia pestis, Neisseria gonorrhea, Chlamydia trachomatis, Chlamydia pneumoniae, Streptococcus pneumoniae, Staphylococcus aureus, group A Streptococcus, group B Streptococcus, Neisseria meningiditis, Haemophilus influenzae, Acinetobacter baumii, Helicobacter pylori and Camphylobacter jejuni.

8. The composition of claim 1 wherein the bacterial strain in (b) is modified to prevent repression of an endogenous immunomodulatory flagellin polypeptide.

9. The composition of claim 8 wherein the bacterial strain is selected from Salmonella typhimurium, Salmonella typhi, Salmonella paratyphi, Salmonella enteriditis, and Listeria monocytogenes.

10. The composition of claim 1 wherein the fusion protein in (d) comprises a cell penetrating polypeptide sequence.

11. The composition of claim 1 wherein the fusion protein in (d) comprises a viral, bacterial or parasite antigen.

12. The composition of claim 1 wherein said immunomodulatory flagellin polypeptide is in single chain form.

13. The composition of claim 1 wherein the immunomodulatory flagellin polypeptide comprises required portions of the D1 domain, the D0 domain or both as a single polypeptide or as separate polypeptides.

14. The composition of claim 1 wherein the innate response augments an adaptive response.

15. A method to induce an innate immune response in a subject which method comprises administering to a subject in need of such induction an effective amount of the composition of claim 1.
